(19) United States
(12) Patent Application Publication
(10) Pub. No.: US 2025/0025062 A1
(43) Pub. Date: Jan. 23, 2025

(54) ELECTRIC FIELD GENERATION

(71) Applicant: HM TECHNOLOGY INTERNATIONAL LIMITED, Middlesex (GB)

(72) Inventor: Hrand Mami MAMIGONIANS, London (GB)

(21) Appl. No.: 18/774,809

(22) Filed: Jul. 16, 2024

(30) Foreign Application Priority Data
Jul. 19, 2023 (GB) 2311057.0

(51) Int. Cl. A61B 5/053; A61B 5/00; G01N 27/02; G01R 27/26; H05K 1/18

(52) U.S. Cl. CPC A61B 5/053; A61B 5/0077; G01N 27/023; G01R 27/2647; H05K 1/181; H05K 2201/10151

(57) ABSTRACT

Penetrating matter (non-organic, organic or biological) with electric fields is shown, to identify one or more properties of the matter. A first electrode (111) is energized and an output signal from a capacitively coupled second electrode (115) is monitored. The output signal is processed to identify properties of the matter (which for biological matter could indicate the presence of cancer). The first electrode and the second electrode are selected from a plurality of electrodes mounted on a first planer surface of a dielectric substrate (401). The electrodes are circumferentially and substantially evenly displaced upon the planer surface around a hole in the substrate.

### CROSS REFERENCE TO RELATED APPLICATIONS

This application claims priority from United Kingdom Patent Application number 2311057.0, filed on Jul. 19, 2023 the whole contents of which are incorporated herein by reference.

### BACKGROUND OF THE INVENTION

The present invention relates to an apparatus for generating electric fields and, in particular, for generating electric fields suitable for penetrating matter to identify one or more properties of said matter. The present invention also relates to a method of penetrating matter with electric fields to identify one or more properties of the matter.

A sensor array for sensing the electrical permittivity of an object is disclosed in U.S. Pat. No. 8,994,383. A dielectric layer presents a surface defining the base of a volume in which a test object is placed. An electrically active layer beneath the dielectric layer is also provided. A first set of electrodes extend in a first direction and a second set of electrodes extend in a second direction that is perpendicular to the first, such that each one of the first set of electrodes intersects with one of the second set of electrodes. An input signal is applied to a first electrode or the first set of electrodes, thereby generating an electric field that extends outside the sensor array and into the volume. Output signals are detected in each one of the second set of electrodes that intersects the first electrode. The output signals are caused by capacitive coupling between the first electrodes and each one of the electrodes in the second set of electrodes, and are indicative of the electrical permittivity of the volume above the intersection of the first electrode and each one of the electrodes of the second set of electrodes.

An array of this type has been considered for detecting properties of organic material and, in particular, human skin to detect skin cancers. GB2577927 describes an application of this type, in which probe calibration may be achieved by scanning a reference region of skin that does not include the skin condition and then deploying the probe against the skin condition to produce test data.

In an environment where medical decisions may be based on an analysis of biological matter, an accurate and reliable testing procedure is required. In this respect, a first problem exists in that when many electrodes are present, of which many are not in actual use for a scanning operation, these electrodes become charged and as such these charges may disrupt the electric fields required for the scanning operation. Furthermore, when making physical contact with biological material and, in particular, a subject's skin, the act of making physical contact may in turn change the attributes of the condition being monitored; thereby resulting in the production of erroneous results.

### BRIEF SUMMARY OF THE INVENTION

According to a first aspect of the present invention, there is provided an apparatus for generating electric fields, wherein said electric fields are suitable for penetrating matter to identify one or more properties of said matter, comprising: a first dielectric substrate presenting a first planar surface and a second planar surface, with a hole having a first radius; a plurality of circumferentially and substantially evenly displaced scanning electrodes on said first planar surface, located at a second radius around said hole; and respective electrical conductors from each said scanning electrode configured to pass through said first dielectric substrate to said second planar surface.

In an embodiment, the apparatus comprises a plurality of circumferentially and substantially evenly displaced secondary electrodes on said first planar surface, wherein: said secondary electrodes are positioned at a third radius; and said third radius is larger than said second radius. Each said secondary electrode is radially aligned with a respective scanning electrode. The dielectric substrate is implemented as a first circuit board; said first circuit board is substantially circular and has a fourth radius that is larger than said third radius.

According to a second aspect of the invention, there is provided a method of generating electric fields for penetrating matter to identify one or more properties of said matter, comprising the steps of: locating an apparatus such that a hole of a first radius in a dielectric substrate is at the position of said matter; and energizing the apparatus to produce said electric fields, wherein: said dielectric substrate presents a first planar surface and a second planar surface; a plurality of circumferentially and substantially evenly displaced scanning electrodes are located on said first planar surface at a second radius around said hole; and respective electrical conductors from each said scanning electrode are configured to pass through said first dielectric substrate to said second planar surface.

Embodiments of the invention will be described, by way of example only, with reference to the accompanying drawings. The detailed embodiments show the best mode known to the inventor and provide support for the invention as claimed. However, they are only exemplary and should not be used to interpret or limit the scope of the claims. Their purpose is to provide a teaching to those skilled in the art. Components and processes distinguished by ordinal phrases such as “first” and “second” do not necessarily define an order or ranking of any sort.

### BRIEF DESCRIPTION OF THE SEVERAL VIEWS OF THE DRAWINGS

FIG. 1 shows an apparatus for generating electric fields;

FIG. 2 shows the introduction of substantially evenly displaced secondary electrodes;

FIG. 3 shows the introduction of a circular hole;

FIG. 4 shows an implementation of the dielectric substrate as a circular circuit board;

FIG. 5 shows the introduction of a second circuit board that is parallel with the first circuit board introduced in FIG. 4;

FIG. 6 shows the first circuit board and a schematic representation of the electronics contained on the second circuit board;

FIG. 7 shows the apparatus located within a housing to facilitate application upon a subject's skin;

FIG. 8 shows an arrangement for examining specimens;

FIG. 9 shows an alternative arrangement for examining specimens;

FIG. 10 shows the arrangement of FIG. 9 with an additional dielectric material;

FIG. 11 shows procedures performed by the processor identified in FIG. 6;

FIG. 12 shows an example of a scanning cycle;

FIG. 13 details procedures for energizing electrodes identified in FIG. 12;

FIG. 14 shows the production of output samples;

FIG. 15 details procedures for identifying and storing information identified in FIG. 13;

FIG. 16 shows a data table;

FIG. 17 shows the apparatus identified in FIG. 7 located in a cradle;

FIG. 18 shows the apparatus of FIG. 17 when viewed in the direction of the arrow identified in FIG. 17;

FIG. 19 illustrates the deployment of the apparatus identified in FIG. 17; and

FIG. 20 shows a further deployment of the apparatus identified in FIG. 17.

### DETAILED DESCRIPTION OF EMBODIMENTS OF THE INVENTION

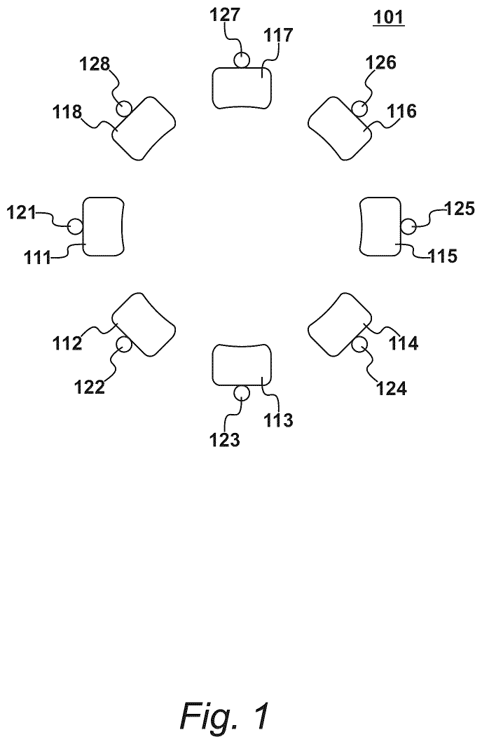

### FIG. 1

An apparatus for generating electric fields is shown in FIG. 1 that is suitable for penetrating matter to identify one or more properties of the matter. The matter could be inorganic, organic or biological. The biological matter could comprise human skin and a test could be performed upon a person to identify cancerous skin for example. Alternatively, the biological material could consist of a specimen removed from a subject for analysis.

A dielectric substrate presents a first planar surface 101 as shown in FIG. 1. Circumferentially and substantially evenly displaced scanning electrodes are attached to the first planar surface 101. In the example shown in FIG. 1, eight scanning electrodes are provided, consisting of a first scanning electrode 111, a second scanning electrode 112, a third scanning electrode 113, a fourth scanning electrode 114, a fifth scanning electrode 115, a sixth scanning electrode 116, a seventh scanning electrode 117 and an eighth scanning electrode 118. In alternative configurations, fewer electrodes may be provided (possibly a total of four) or more electrodes may be provided, such as sixteen.

Each scanning electrode includes an electrical conductor, comprising a first conductor 121 to an eighth conductor 128, attached to the first scanning electrode 111 to the eighth scanning electrode 118 respectively, which pass through the dielectric substrate to a second a planar surface on the opposite side of the substrate. In this way, it is possible for the scanning electrodes 111 to 128 to be energized or monitored, while minimizing the presence of additional conductors on the first planar surface, which could become polarized and thereby introduce errors.

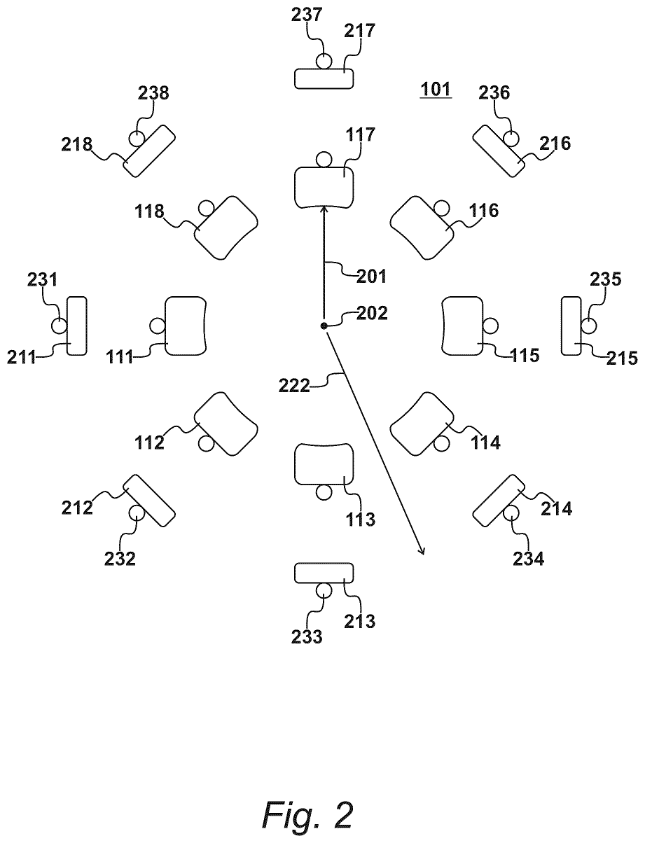

### FIG. 2

In an embodiment, as illustrated in FIG. 2, circumferentially and substantially evenly displaced secondary electrodes are also introduced to the first planar surface 101. The scanning electrodes 111 to 118 are positioned at a first radius 201 from a central point 202. The secondary electrodes comprise a first secondary electrode 211, a second secondary electrode 212, a third secondary electrode 213, a fourth secondary electrode 214, a fifth secondary electrode 215, a sixth secondary electrode 216, a seventh secondary electrode 217 and an eighth secondary electrode 218. The secondary electrodes are positioned at a second radius 222 from the central point 202 and the second radius 222 is larger than the first radius 201.

In the embodiment of FIG. 2, each secondary electrode (electrode 211 for example) is radially aligned with a respective scanning electrode (111).

Each secondary electrode 211 to 218 also includes a secondary electrical conductor, identified as secondary electrical conductors 231 to 238 respectively. These pass through the dielectric substrate to the second planar surface on the opposite side of the substrate.

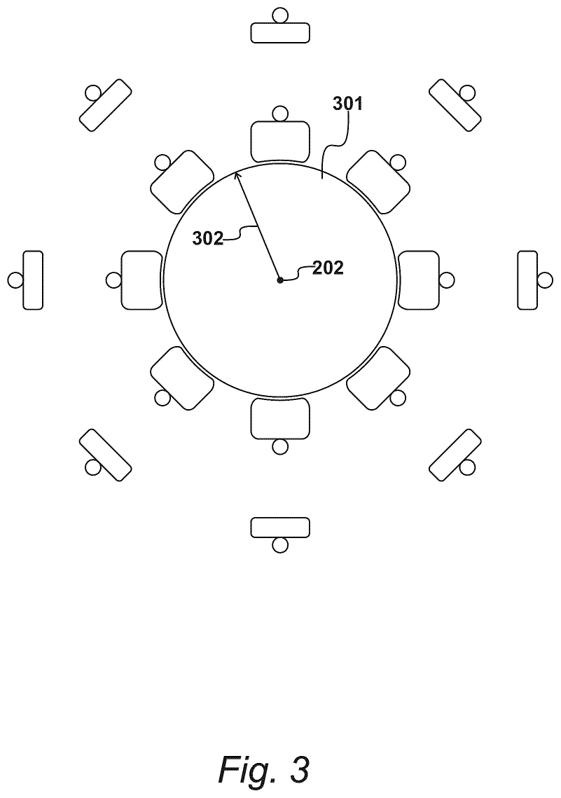

### FIG. 3

In accordance with an aspect of the invention, a circular hole 301 is cut in the first dielectric substrate, which has a third radius 302 from the central point 202, that is smaller than the first radius 201.

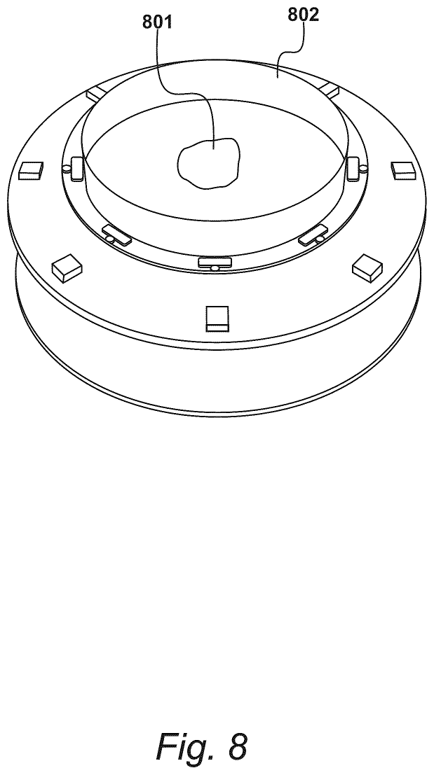

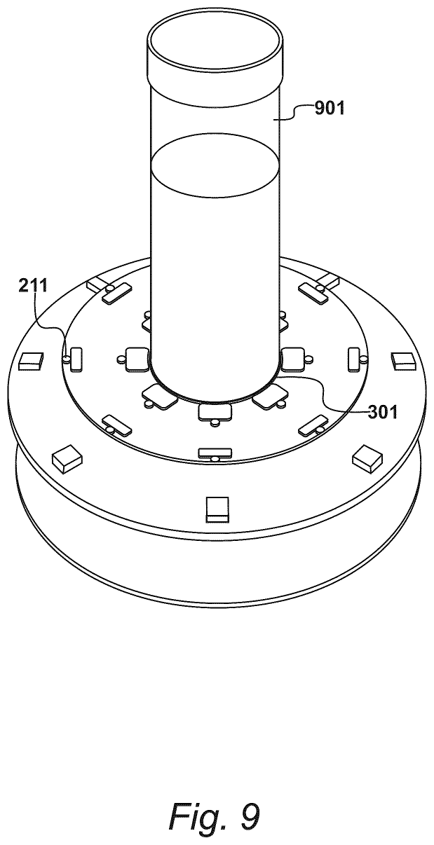

Electric fields radiate away from this hole 301 in three-dimensional space. When considering a skin condition, a housing containing the apparatus may be located upon a subject, such that the skin condition itself occupies the region where the hole is present. As such, no physical electrode contact is made upon the actual skin condition being investigated, during scanning operations in which electric fields penetrate the skin condition. Where contact is made with the skin, around the skin condition of interest, the electrodes are electrically insulated. In an embodiment, this insulation is provided by a glass cover. Over the position of the circular hole, the glass cover is transparent to facilitate optical viewing of the skin condition by a clinician. Around the hole, at the position of the electrodes, the glass cover may be opaque. The hole may also be used to receive a receptacle, such as a tube, as described with reference to FIG. 8 and FIG. 9. The receptacle may be of a standard type used for storing specimens in formalin (methanal in aqueous solution) and examination may take place with the formalin present.

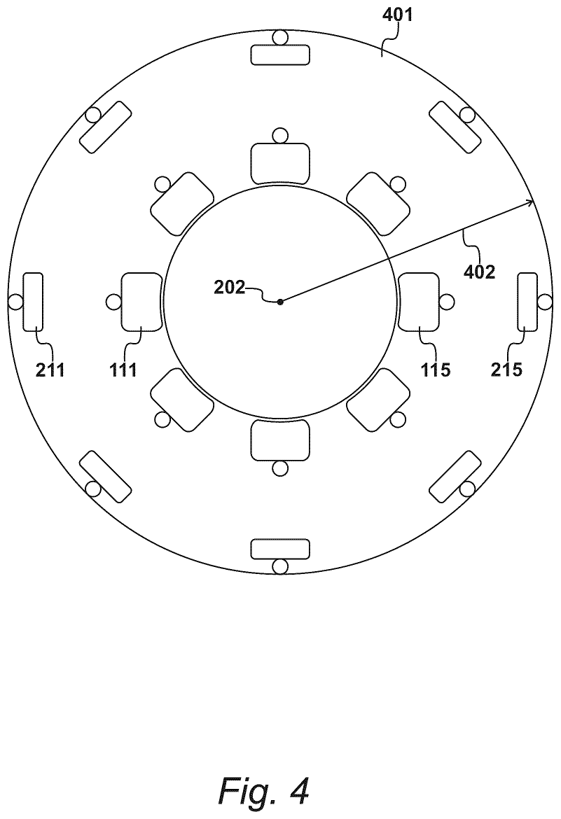

### FIG. 4

In an embodiment, the dielectric substrate 101 is implemented as a first circuit board 401 which is circular and has a fourth radius 402 from the central point 202 that is larger than the second radius 222. Thus, in this embodiment, the electrical conductors (121-128) from the primary electrodes, and similar electrical conductors (231-238) from the secondary electrodes, pass through the circuit board to present electrical connections on the opposite side of the circuit board to that shown in FIG. 4.

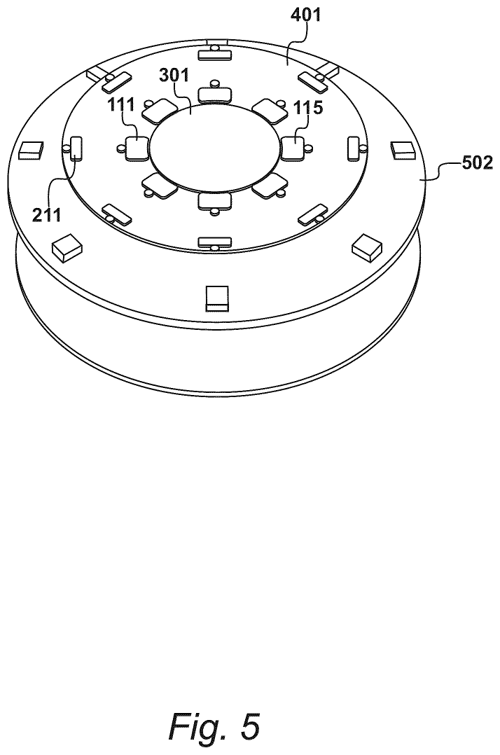

### FIG. 5

The first circuit board 401 is also shown in FIG. 5. In addition, there is provided a second circuit board 502 that is parallel with the first circuit board 401. In this embodiment, the second circuit board is also circular and has a radius that is greater than the radius 402 of the first circuit board. In an alternative embodiment, the second circuit board is of a similar dimension and this approach may be beneficial when reducing overall size for a hand-held unit.

The second circuit board 502 includes electronics for energizing and monitoring the scanning electrodes (111-118), and for changing the properties of the secondary electrodes (211-218). The electronics also includes data storage devices for the storage of data and executable instructions controlling the operation of the device, In an embodiment, executable instructions may be updated from external sources and as such may be identified a firmware.

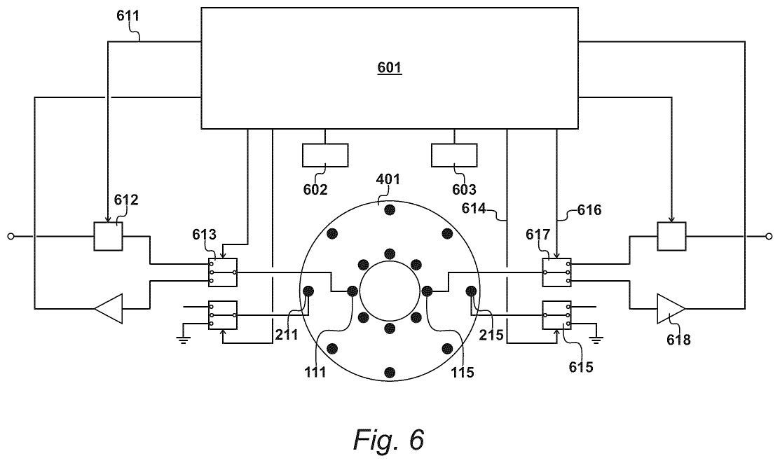

### FIG. 6

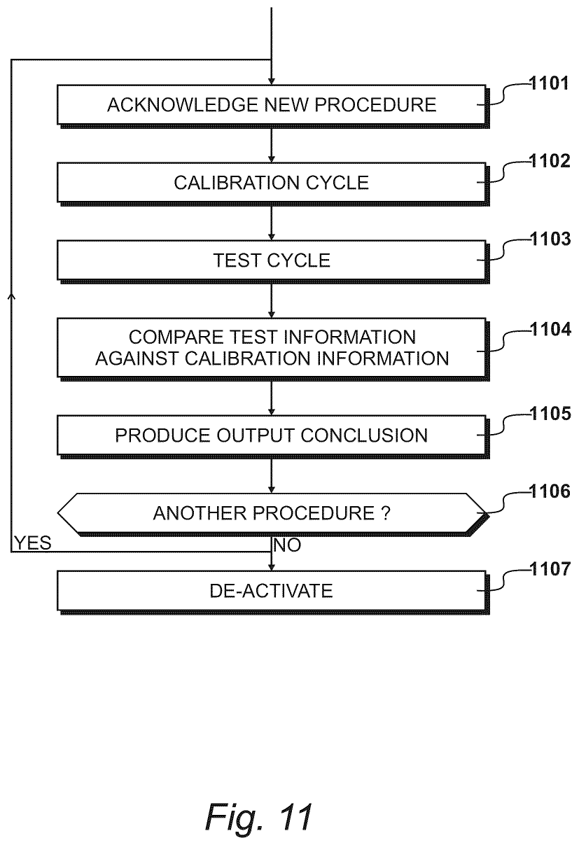

A schematic representation of the first circuit board 401 is shown in FIG. 6, along with a representation of the electronics contained on the second circuit board 502. The electronics includes a processor 601 and operations performed by the processor 601 will be described with reference to FIG. 11 to FIG. 16.

An embodiment includes light-emitting devices 602 for illuminating the apparatus and a lens, thereby enhancing a clinician's ability to view the area under examination. These components are particularly useful when examining skin and allow the clinician to compare electrical characteristics (permittivity and conductivity) against visual images. In an embodiment, the conductivity and permittivity data derived from the scanning operations may be displayed to a clinician on a suitable display device such as a laptop computer.

FIG. 6 shows electronics relevant to the first scanning electrode 111, the first secondary electrode 211, the fifth scanning electrode 115 and the fifth secondary electrode 215. However, it should be appreciated that this electronics is repeated, such that all of the scanning electrodes and all of the secondary electrodes are connected in a similar way.

The electronics shown in FIG. 6 allows the first scanning electrode 111 to be energized while the fifth scanning electrode 115 is monitored.

Alternatively, it also provides for the fifth scanning electrode 115 to be energized while the first scanning electrode 111 is monitored. In addition, both the first secondary electrode 211 and the fifth secondary electrode 215 may be connected to ground or may be allowed to float (open circuit). Furthermore, in an alternative embodiment, a connection to ground may be made via a variable resistor, such that intermediate resistivity values may be established and additional intermediate data values may be obtained. In an embodiment, all of the non-selected scanning electrodes float; that is to say, they are not connected to ground as this will tend to attract the electric field away from the monitoring scanning electrode. Thus, in this way, the electric field is focussed towards the monitoring electrode.

For the purposes of this description, it will be assumed that the first electrode 211 is being energized while the fifth electrode 115 is being monitored. In this embodiment, two energizations take place: for the first, the fifth secondary electrode is grounded and for the second the fifth secondary electrode floats. The first secondary electrode is not adjusted for this operation and is allowed to float. In an alternative embodiment, more than two energizations would take place.

The processor 601 provides an energizing control signal on a first control line 611 to a first switch 612. The switch receives as its input a constant scanning voltage which is in turn supplied to a second switch 613. When activated, the first switch 612 sends an energizing strobing pulse to the first scanning electrode 111 via the second switch 613. The first scanning electrode 111 is capacitively coupled to the fifth scanning electrode 115 that is being monitored. The electric field enters matter placed on the first circuit board 401 along many paths that follow a three-dimensional trajectory having components that are perpendicular to the plane of FIG. 6. The duration of the analogue strobing pulse is determined by the first control signal under the control of the processor 601.

A second control line 614 activates a third switch 615 that, on a first energizing operation, connects the fifth secondary electrode 215 to ground. As a result of this, the electric field is also attracted to this grounded electrode; thereby changing the distribution of the electric field in three-dimensional space. Furthermore, a third control line 616 operates a fourth switch 617, such that the monitored signal is supplied to the processor 601 via an amplifier 618.

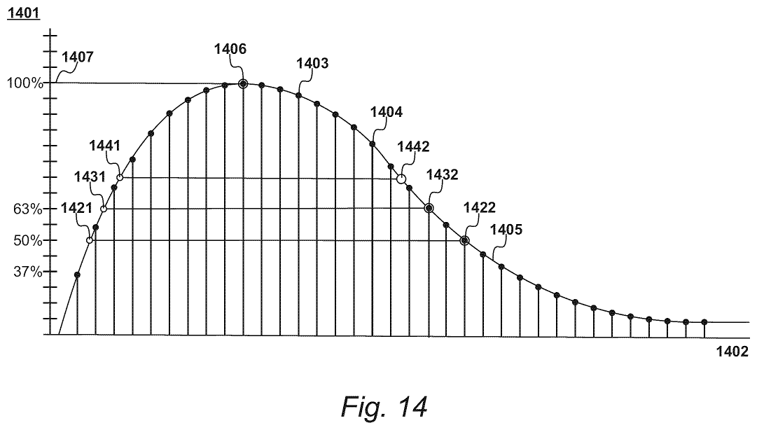

An analogue output signal from the amplifier 618 is sampled, as described with reference to FIG. 14, and a further scanning operation is then performed, after operation of the third switch 615; such that the fifth secondary electrode 215 is allowed to float. During this second scanning operation, the electric field is less attracted to the fifth secondary electrode, which results in a different distribution of the electric field within three-dimensional space.

These two scanning operations, using the same two capacitively connected scanning electrodes, obtain different results; particularly given that differing levels of penetration will occur. Thus, by considering these two different levels of penetration, it is possible to determine the extent of changing characteristics of the matter under examination at different depths. This can be particularly useful when considering skin cancer for example, given that if an anomaly is present, an indication can be derived as to the extent of its depth and as such provide an indication of greater or lesser threat.

In an embodiment, a significant amount of data is collected, given that it is possible for each of the scanning electrodes (of which there are eight in the embodiment of FIG. 6) to be selected as an energized electrode. Furthermore, for each energized electrode, all of the remaining (seven in this embodiment) electrodes may be considered as a monitored electrode. Thus, a total of fifty-six combinations are possible (for this particular embodiment) and a scanning operation is performed twice for each combination, thereby giving a total of one hundred and twelve datasets for each cycle.

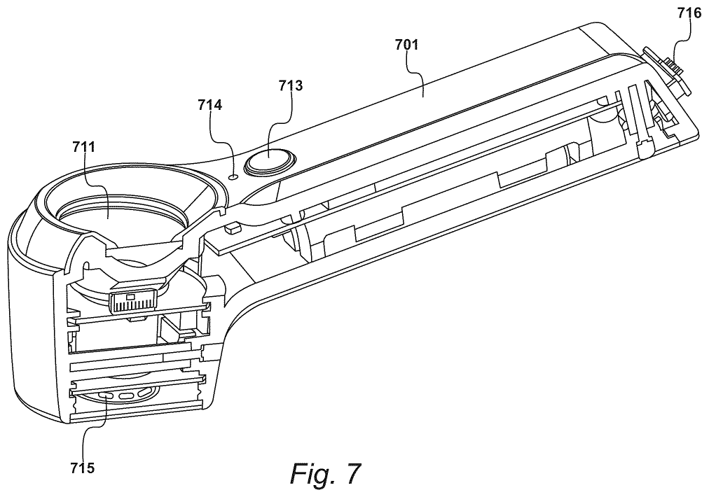

### FIG. 7

The presence of circular hole 301 makes the apparatus described with reference to FIG. 5 particularly suitable for examining skin conditions. A suspicious skin condition may be identified that requires further tests to determine whether further examination and treatment are required.

When using electric-field-generating electrodes, it is undesirable for an airgap to exist between the skin and the electrodes. However, if the electrodes are brought into contact with the skin condition itself, this can change characteristics of the skin and thereby produce erroneous results. Thus, the configuration described with reference to FIG. 5 allows the electrodes to be brought into contact with the skin, thereby avoiding the introduction of an airgap, but not in contact with the actual skin condition being examined.

It is not necessary, nor desirable, for the conducting electrodes to be in direct contact with the skin. In an embodiment, a glass plate is provided that makes physical contact with the skin. This ensures that there is no airgap and maintains consistent positioning of the electrodes from the skin. The apparatus does not conduct an electrical current into the skin. The apparatus creates an electric field that penetrates the glass cover and the skin tissue.

The circular hole 301 surrounds the skin condition along with the scanning electrodes 112 to 118 and the secondary electrodes 211 to 218. Thus, in a deployment for the examination of skin, the apparatus described with reference to FIG. 5 is effectively inverted.

In an embodiment, for the examination of skin conditions, it is possible for the apparatus described with reference to FIG. 5 to be located within a housing 701 to facilitate application upon a subject's skin. In this embodiment, the hole 301 is extended through the apparatus allowing a clinician to make a visual examination prior to aligning the skin condition for the electric field examination. In an embodiment, the hole is capped with a lens at a viewing end, to enhance visibility, and capped by a glass plate at the contact end. The embodiment of FIG. 7 includes a lens 711 to facilitate visual inspection. In an embodiment, the lens at the top and the glass plate at the bottom effectively seals the apparatus to prevent fluid entry.

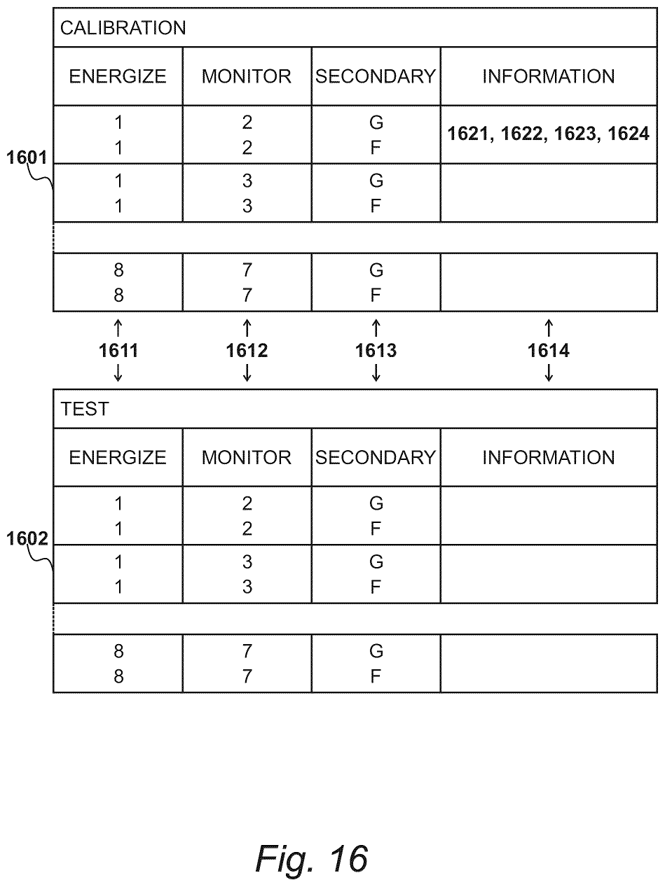

To produce meaningful results, it is necessary for the data generated while testing to be compared against calibration data. For the apparatus shown in FIG. 7, the apparatus may firstly be located upon an area of healthy skin which does not show any evidence of a skin condition existing. In this position, a full scanning cycle is performed to produce calibration data, as described with reference to FIG. 14, which is then processed to generate calibration information as described with reference to FIG. 16. Having performed the calibration cycle, the housing 701 is then brought to the position of the skin condition, such that a testing cycle is performed that generates test data and test information that are similar to the calibration data and calibration information. In alternative arrangements, the calibration data may be produced while the apparatus is open to ambient air.

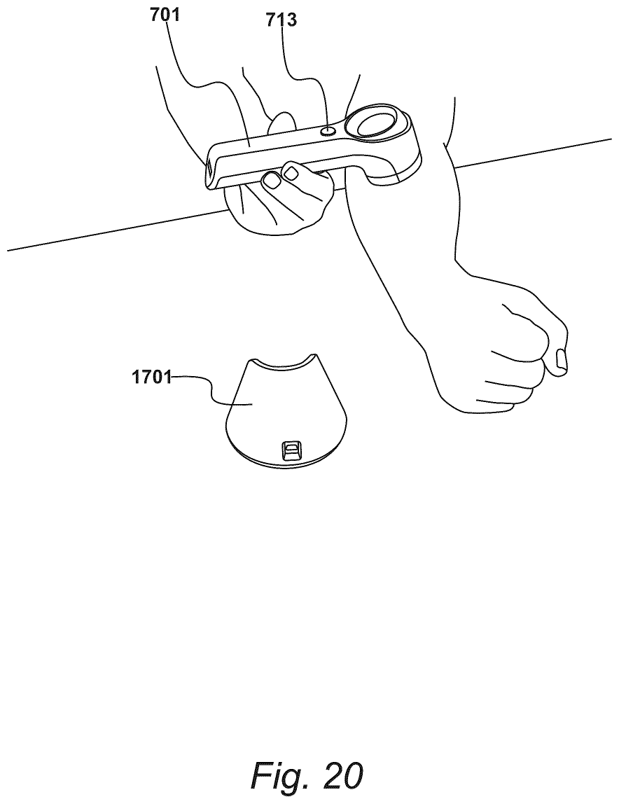

The apparatus shown in FIG. 7 may communicate wirelessly with processing equipment such as a laptop computer. A first scanning cycle, to produce reference data, is initiated by applying pressure to a button 713. In response to this, a light emitting indicator 714 emits light of a different colour to indicate that a scanning cycle is in progress. At the end of the scanning cycle, the colour of light emitted changes back to its passive colour or alternatively may not be illuminated at all. The apparatus is then deployed, as described with reference to FIG. 20, to produce test data and a scanning procedure for the generation of test data is again initiated by applying pressure to button 713.

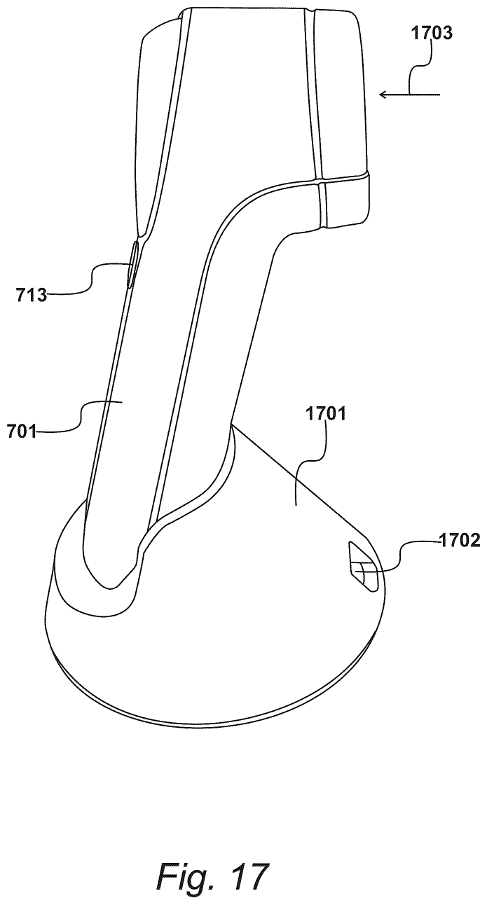

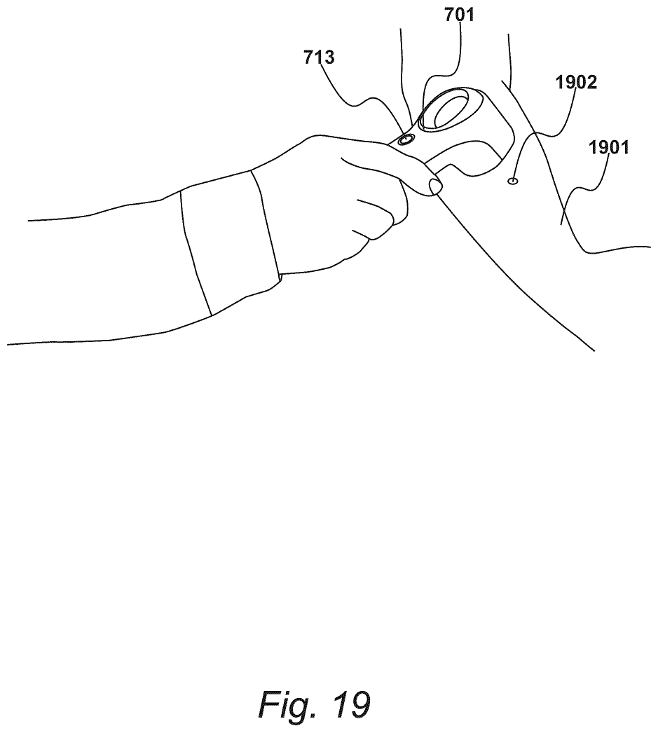

To enhance visual inspection, as described with reference to FIG. 19, the area being inspected is illuminated by light emitting devices 715. To conserve power, light emitting devices 715 are usually not energized. In an embodiment, the LED devices 715 are not energized when the apparatus is located within a charging cradle, as described with reference to FIG. 17. Upon removing the apparatus 701 from a cradle, the light emitting devices 715 are energized to facilitate visual observation and also to indicate to a user that the probe is ready to perform scanning operations.

A plug 716 extends from the bottom of the probe housing to facilitate the charging of internal batteries when the apparatus is secured within a cradle. Furthermore, as an alternative to transmitting data wirelessly, data communication may take place by storing data within the apparatus and then effecting a download of data when the apparatus is returned to a cradle.

Data communication with the apparatus may also be performed to perform firmware updates, either via a connected laptop computer or directly via a wireless connection.

### FIG. 8

The arrangement described with reference to FIG. 5 may also be deployed for examining specimens. Thus, in a clinical situation, it is possible for a biopsy to be taken and for analysis to be performed immediately, as an alternative to, or in addition to, sending a sample for laboratory testing.

In the embodiment shown in FIG. 8, a specimen 801 is placed in a container 802 surround by an appropriate electrically-inert liquid. As shown in FIG. 8, the container 108 is located upon the first planar surface 101. The scanning electrodes 111 to 118 are energized and monitored as previously described. Alternatively, specimens can be examined as received in containers of formalin.

When examining specimens as shown in FIG. 8, it is also necessary to perform a calibration cycle before the actual testing of the specimen is performed. In this example, calibration data is generated by performing a complete scanning operation without the container 802 being present. Thus, the calibration data effectively represents characteristics of the ambient air. Alternatively, a similar container containing formalin but with no specimen may be used to create the reference data.

### FIG. 9

As an alternative to placing a wide container upon the planar surface, as described with reference to FIG. 8, it is possible for a tube 901 to be located within the circular hole 301. Advantages of this approach are that the specimen itself requires less surrounding liquid and the tube is held firmly in position while a scanning operation is performed. However, compared to the arrangement described with reference to FIG. 8, the secondary electrodes 211 to 218 will have less influence.

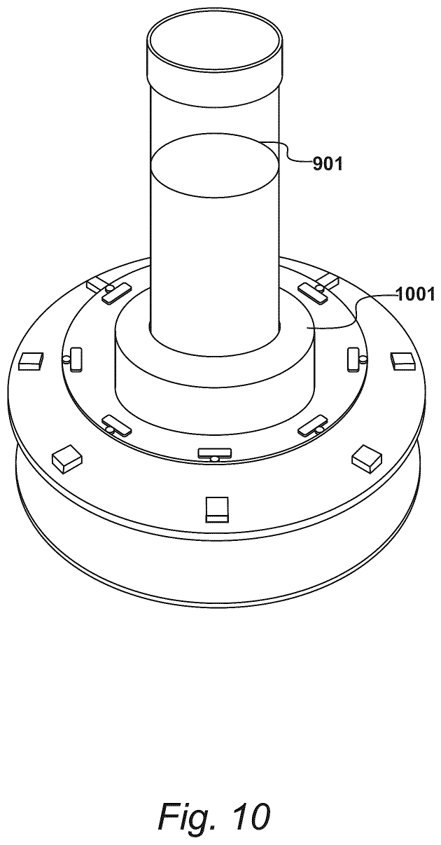

### FIG. 10

Experiments have shown that it is also possible to place a ring 1001 around the tube 901 to modify the concentration of electric fields. In an embodiment, a ring 1001 is made from a dielectric material such that, after a few scanning operations, the material becomes charged and as such, the presence of this charge influences the electric fields generated by the scanning electrodes.

### FIG. 11

Procedures performed by the processor 601 when deploying a skin examination apparatus, as described with reference to FIG. 7, or when deployed for a specimen analysis as described with reference to FIG. 8 to FIG. 10, are shown in FIG. 11. At step 1101, the processor acknowledges that a new procedure is to be conducted and appropriately coloured light emitting devices may be illuminated or a message may be presented on a connected laptop computer. Prior to this, the processor 601 will have checked that information from a previous procedure has been transferred and that working memory may now be overwritten.

At step 1102, a calibration cycle is performed and at step 1103 a test cycle is performed. These scanning procedures for both the calibration cycle and the test cycle are substantially similar and the fundamental difference relates to what is actually being examined. Thus, for the apparatus described with reference to FIG. 7, calibration may be performed upon healthy skin, whereafter a test cycle is performed in relation to the potential skin condition.

The calibration cycle and the test cycle both generate a significant amount of data as described with reference to FIG. 14. This data is analysed to produce a smaller volume of information as described with reference to FIG. 16; for both the calibration cycle and the test cycle. At step 1104, the test information is compared against the calibration information and an output conclusion is generated at step 1105.

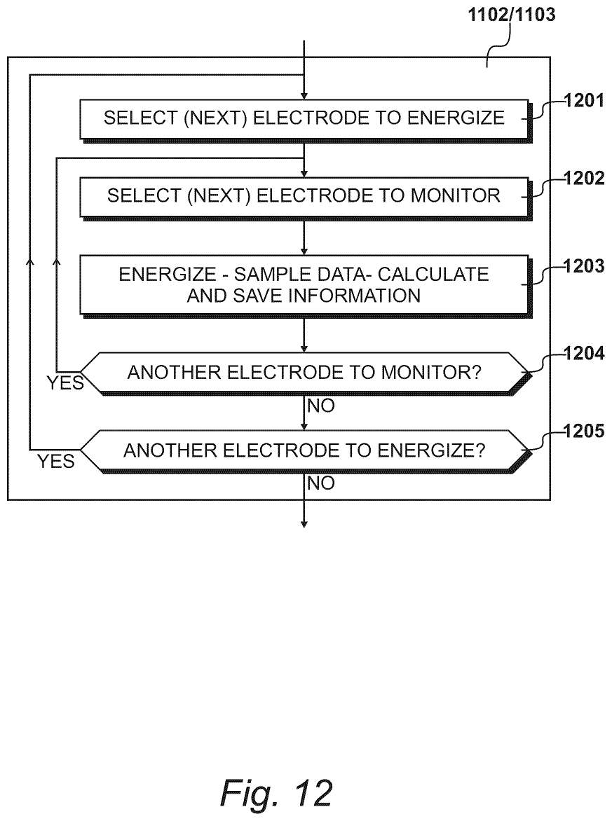

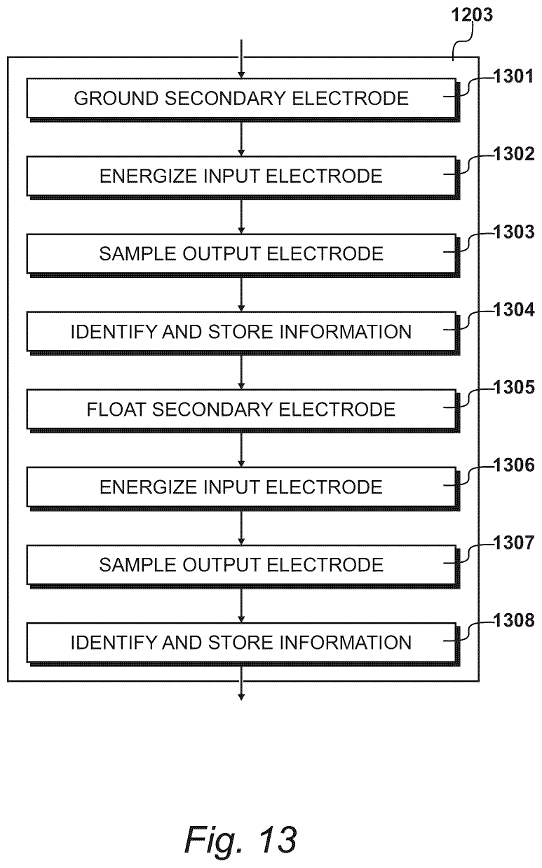

In this embodiment, it is assumed that the device is totally self-contained and is in a position to produce local output. In alternative embodiments, a graphical user interface is provided to a user by means of a laptop computer which may receive the output conclusion, partially-processed information or the raw data, such that some of the procedures described with reference to FIG. 12 and FIG. 13 may be performed on the laptop computer, as an alternative to being performed fully within processor 601.

After producing the output conclusion at step 1105, a question is asked as to whether a new procedure is to be performed and when answered in the affirmative, this position is again acknowledged at step 1101. Alternatively, if the question asked at step 1106 is answered in the negative, the system is deactivated at step 1107.

### FIG. 12

An example of a scanning cycle is shown in FIG. 12. A substantially similar cycle is performed for both the calibration cycle 1102 and the test cycle 1103. At step 1201, a scanning electrode is selected to be energized which, for the purposes of this example, could be the first scanning electrode 111.

At step 1202, an electrode is selected to be monitored which, for the purpose of this example, may be the second scanning electrode 112.

At step 1203, the electrode selected at step 1201 is energized and the electrode selected at step 1202 is monitored. Data is saved and calculations are performed to generate information that is also saved.

At step 1204, a question is asked as to whether there is another electrode to monitor which, when answered in the affirmative, results in the next electrode being selected at step 1202. Thus, in this example, the first scanning electrode 111 is energized again with the third scanning electrode 113 being monitored.

Thus, the question asked at step 1204 will continue to be answered in the affirmative until all of the remaining electrodes have been selected as an electrode to be monitored. When the question asked at step 1204 is answered in the negative, a question is asked at step 1205 as to whether another electrode is present to be energized. Thus, on a first iteration, the question asked at step 1205 will be answered in the affirmative and the next electrode will be selected at step 1201. Thus, for the purpose of this example, the second scanning electrode 112 may be selected at step 1201 with the third scanning electrode 113 being selected at step 1202.

Again, eventually, the question asked at step 1204 will be answered in the negative and the next electrode will be selected to be monitored. As this continues, the question asked at step 1205 will be answered in the negative when all of the electrodes present have been selected at step 1201.

The procedure described with reference to FIG. 12 ensures that every possible combination of energizing electrodes and monitoring electrodes has been considered. The sequential ordering described is attractive given that it allows simple counters to be incremented. However, fundamentally, the selection of energizing and monitoring electrodes may take place in any order. In an embodiment, non-selected scanning electrodes are not grounded. However, these will tend to attract the electric field away from the monitoring electrode. Thus, in an alternative embodiment, the non-selected scanning electrodes float to achieve improved electric field focussing.

### FIG. 13

Procedures 1203 for energizing the electrodes, identified in FIG. 12, are detailed in FIG. 13. An electrode to be energized has been selected and an electrode to be monitored has been selected.

At step 1301, secondary electrode 215 is grounded. At step 1302, the input electrode 111 is energized and at step 1303 an output signal from the monitored electrode 115 is sampled. In an embodiment, each energizing pulse lasts for a duration of ten micro-seconds (10 μs) and individual pulses are separated by a duration of ninety-microseconds (90 μs). During each cycle, one hundred samples are taken, which requires a sampling rate of five mega-Hertz (5 MHZ).

An analogue-to-digital converter, forming part of the processor 601, converts each sample into a twelve-bit representation and as a result of this, each energizing pulse generates a significant amount of data. However, the processor 602 is fast enough to allow a significant amount of processing to take place during the sample period. Thus, by comparing samples, it is possible to identify a peak value and the regular intervals between samples allows the time at which this peak value occurred to be determined. Thus, each sample point is made up of data that defines a voltage level at a particular time.

This raw data is saved at step 1303. The raw data is then processed to produce a smaller volume of information that is stored at step 1304. At step 1305, the fourth switch 617 is operated, such that the fifth secondary electrode 215 now floats.

The procedures identified above are now repeated. Thus, at step 1306, the input electrode 111 is energized and at step 1307 an output from the monitored electrode is sampled. Information is then identified and stored at step 1308.

### FIG. 14

The production of output samples at step 1303, or at step 1307, is illustrated in FIG. 14. In the representation of FIG. 14, output voltage 1401 is plotted against time 1402. The sampling operation creates data points, such as data point 1403 and data point 1404. As is well known in the art, it is also possible to fit a curve 1405 to the existing data points, such that values on this curve may be calculated to a higher degree of accuracy through a process of interpolation. From this, it is possible to identify a peak value 1406.

Having calculated the peak value 1406, it is then possible to identify points at which a proportion of this peak value has been presented. Thus, from the large volume of raw data, it is possible to calculate a much-limited volume of information which conveys what is required in terms of a peak and a rate of discharge. In particular, the absolute peak value and the rate of discharge are related to the conductivity and permittivity of the material.

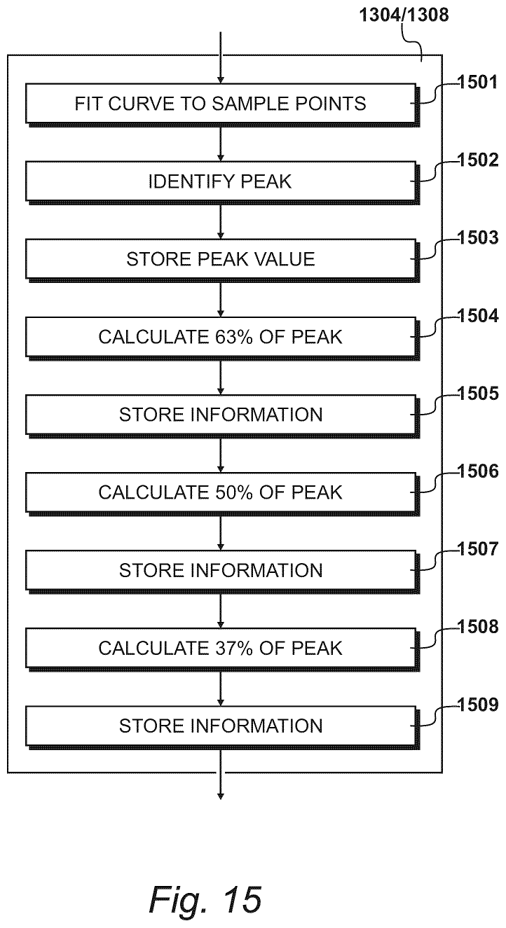

### FIG. 15

Procedures 1304 and 1308 for identifying and storing information are shown in FIG. 15. At step 1501, a mathematical representation of curve 1405 is calculated in an exercise of fitting sample points 1403, 1404 etc to a mathematically defined curve.

At step 1502, the peak 1406 is identified and this peak information 1407 is stored at step 1503.

At step 1504, a value is calculated that represents sixty-three percent (63%) of the peak value 1407.

At step 1505, information is identified, consisting of a first data point 1421 and a second data point 1422 at which the curve 1405 passes through the sixty-three percent value.

At step 1506, a value is calculated that represents fifty percent (50%) of the peak value 1407. A first data point 1431 and a second data point 1432 are identified where the curve 1405 crosses this fifty percent level.

At step 1508, a value is calculated that represents thirty-seven percent (37%) of the peak value 1407. Again, at step 1509, a first data point 1441 is calculated, along with a second data point 1442 showing where the curve 1405 crosses these values.

### FIG. 16

The information calculated at steps 1502, 1504, 1506 and 1508 is stored in a database and a representation of this database is illustrated in FIG. 16.

A data table 1601 is constructed for the calibration data and a similar data table 1602 is constructed for the test data. In a first column 1611, the electrode being energized is recorded and in a second column 1612 the electrode being monitored is recorded. For each of these combinations, a third column 1613 records whether the relevant secondary electrode was grounded or allowed to float. The resulting information is then stored in a fourth column 1614.

In this embodiment, the information represents the first peak value 1621. The information then represents the two positions for thirty-seven percent of the peak value 1622, the two positions for fifty percent of the peak value 1623 and the two positions for sixty-three percent of the peak value 1624.

After fully populating the database table of FIG. 16, the resulting information consists of a relatively small volume compared to the totality of raw data generated through the scanning and sampling operations.

In an embodiment, this information is transferred to a laptop computer for subsequent processing. The overall objective is to identify the nature of the matter under consideration. In particular, when scanning skin conditions, the overall objective is to give an indication as to whether the skin condition is considered benign or whether the skin condition is considered malignant and therefore requires further attention.

As is clear from the data table of FIG. 16, similar combinations exist for both the calibration stage and the test stage. Thus, specific similar information entries may be directly compared to determine the extent to which they differ. Thus, a larger difference may indicate that skin under test, for example, has characteristics that differ significantly to healthy skin and therefore further investigation should be prompted.

Sophisticated algorithms can be developed for analysing the information generated. For example, extreme values may be removed and weightings may be given for particular electrode combinations. Thus, data derived from lower levels of penetration may be subtracted from data derived from higher levels of penetration to focus on these deeper penetration levels. In addition to or as an alternative, many data sets may be generated and tested by alternative means as a mechanism for training a machine learning system.

### FIG. 17

The apparatus described with reference to FIGS. 1 to 16 facilitates the deployment of a method of generating electric fields for penetrating matter to identify one or more properties of this matter. The method may comprise the steps of locating an apparatus such that a hole of a first radius in a dielectric substrate is at the position of the matter for which properties are to be determined. The apparatus is energized to produce electric fields. The dielectric substrate presents a first planar surface and a second planar surface along with a plurality of circumferentially and substantially evenly displaced scanning electrodes which are located on the first planar surface at a second radius around the hole. Respective electrical conductors for each of the scanning electrodes are configured to pass through the first dielectric substrate to the second planar surface. The apparatus may be deployed within a housing for supporting the dielectric substrate upon a subject's skin and may additionally comprise the step of optically viewing the subject's skin through the hole in the dielectric substrate before initiating the scanning procedures. As described with reference to FIG. 7, the viewing of the subject's skin may be facilitated by the presence of optical lens 711, supported within the housing 701.

The housing 701, is shown in FIG. 17 located within a cradle 1701. The cradle 1701 includes a socket 1702 for physically connecting the apparatus, via the cradle, to a laptop computer or similar device. In addition to downloading data (or information as previously defined), the cradle connection to a laptop computer (or similar device) can also be used to upload firmware updates and re-charge the device.

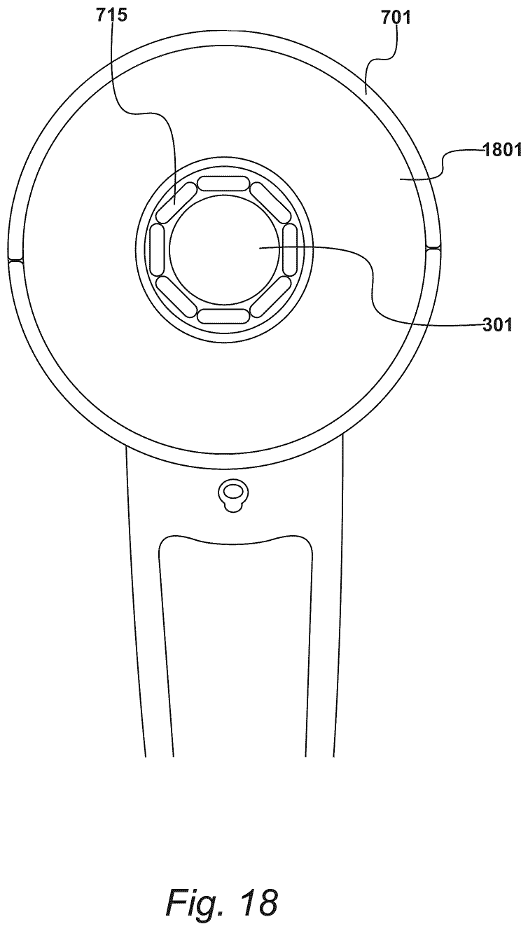

### FIG. 18

The housing 701 as seen when viewed in the direction of arrow 1703 is shown in FIG. 18. The housing 701 supports the light emitting devices 715 and these, along with the hole 301 are visible through a transparent portion of a glass cover. The electrodes are obscured by a coloured portion 1801 of the glass cover.

### FIG. 19

The arm 1901 of a subject is shown in FIG. 19. The skin of arm 1901 includes a region of concern 1902, possibly in the form of a mole. The light emitting devices 715 facilitate optical viewing of the region of concern by a clinician. Thus, the step of viewing the subject's skin is facilitated by illuminating the skin by means of one or more illuminating devices 715 that are supported within the housing 701.

### FIG. 20

After optically viewing the area of interest 1902, the apparatus 701 is brought into contact with the subject's skin, as shown in FIG. 20. However, the hole 301 is located at the position of interest 1902 such that the electrodes are in contact with the skin, via the glass cover, but not at the actual position of interest.

As previously described, the button 713 will have been activated to produce reference data. In the position illustrated in FIG. 20, button 713 is activated again to perform a scanning cycle to produce test data. As described with reference to FIG. 7, after the scanning cycle has completed, the colour of light emitting device 714 will change, thereby indicating to an operative that the scanning cycle has completed. The operative may then return the apparatus to the cradle 1701.

What we claim is:

1. An apparatus for generating electric fields, wherein said electric fields are suitable for penetrating matter to identify one or more properties of said matter, comprising:
a first dielectric substrate presenting a first planar surface and a second planar surface, with a hole having a first radius; and
a plurality of circumferentially and substantially evenly displaced scanning electrodes on said first planar surface, located at a second radius around said hole, wherein:
respective electrical conductors from each of said scanning electrodes configured to pass through said first dielectric substrate to said second planar surface.

2. The apparatus of claim 1, comprising a plurality of circumferentially and substantially evenly displaced secondary electrodes on said first planar surface, wherein:
said secondary electrodes are positioned at a third radius; and
said third radius is larger than said second radius.

3. The apparatus of claim 2, wherein each of said secondary electrodes is radially aligned with a respective one of said scanning electrodes.

4. The apparatus of claim 2, wherein:
said first dielectric substrate is implemented as a first circuit board; and
said first circuit board is substantially circular and has a fourth radius that is larger than said third radius.

5. The apparatus of claim 4, comprising a second circuit board, wherein:
said second circuit board is substantially parallel with said first circuit board; and
said second circuit board comprises electronics for energizing and monitoring said scanning electrodes.

6. The apparatus of claim 5, wherein said second circuit board comprises electronics for introducing a respective resistance between ground and each of said secondary electrodes.

7. The apparatus of claim 4, comprising a container for receiving a specimen, wherein said container is locatable upon said first planar surface.

8. The apparatus of claim 4, comprising:
a tube for receiving a specimen, wherein said tube is locatable within said hole; and
a ring of dielectric material for surrounding said tube, wherein said ring is configured to concentrate said electric fields within said tube, after becoming electrically charged.

9. The apparatus of claim 4, comprising a housing for supporting said apparatus to facilitate application upon skin of a subject, wherein said housing is configured to support one or more lenses to facilitate optical viewing of said skin through said hole in said first dielectric substrate.

10. The apparatus of claim 9, comprising one or more illuminating devices supported within said housing and configured to illuminate an area of said skin being optically viewed.

11. A method of generating electric fields for penetrating matter to identify one or more properties of said matter, comprising steps of:
locating an apparatus such that a hole of a first radius in a dielectric substrate is at a position of said matter; and
energizing said apparatus to produce said electric fields, wherein:
said dielectric substrate presents a first planar surface and a second planar surface;
a plurality of circumferentially and substantially evenly displaced scanning electrodes are located on said first planar surface at a second radius around said hole; and
respective electrical conductors from each of said scanning electrodes are configured to pass through said dielectric substrate to said second planar surface.

12. The method of claim 11, wherein said apparatus comprises a housing for supporting said dielectric substrate upon skin of a subject, the method comprising a step of optically viewing said skin of said subject through said hole in said dielectric substrate.

13. The method of claim 12, wherein said step of viewing said skin of said subject is facilitated by a presence of one or more optical lenses supported by said housing.

14. The method of claim 12, wherein said step of viewing said skin of said subject is facilitated by illuminating said skin by means of one or more illuminating devices supported within said housing.

15. The method of claim 11, wherein said energizing step comprises steps of:
energizing a first scanning electrode of said scanning electrodes;
monitoring an output signal from a capacitively coupled second scanning electrode of said scanning electrodes; and
disconnecting remaining scanning electrodes of said scanning electrodes, such that said remaining scanning electrodes do not provide a conduction path to ground.

16. The method of claim 15, wherein said energizing step comprises steps of:
selecting each of said scanning electrodes as a first energized electrode; and
for each selected energized scanning electrode, selecting each of said remaining scanning electrodes as a second monitored electrode.

17. The method of claim 11, comprising steps of:
energizing a first selected scanning electrode of said scanning electrodes and monitoring a second selected scanning electrode of said scanning electrodes while adjusting electrical characteristics of a secondary electrode radially displaced from said second selected scanning electrode.

18. The method of claim 17, wherein said step of adjusting electrical characteristics comprises introducing resistances between ground and each of said secondary electrodes.

19. The method of claim 15, comprising steps of:
storing a plurality of monitored output signals; and
analysing said stored output signals to identify a peak value.

20. The method of claim 19, comprising a step of assessing an interval between a monitored output signal of said monitored output signals going above a predetermined level and then returning below said predetermined level.
